(19) United States
(12) Patent Application Publication
(10) Pub. No.: US 2003/0068364 A1
(43) Pub. Date: Apr. 10, 2003

(54) Pro-liposomal encapsulated preparations (iv)

(76) Inventors: Josep Garces, Martorell Barcelona (ES); Josep-Lluis Viladot-Petit, Barcelona (ES)

Correspondence Address: COGNIS CORPORATION, 2500 RENAISSANCE BLVD., SUITE 200, GULPH MILLS, PA, 19406

(21) Appl. No.: 10/239,010

(22) PCT Filed: Mar. 17, 2001
(86) PCT No.: PCT/EP01/03082

(30) Foreign Application Priority Data
Mar. 28, 2000 (EP) 001066607.5

(51) Int. Cl. A61K 48/00; A61K 9/127; A61K 31/355; A61K 31/375; A61K 35/78

(52) U.S. Cl. 424/450; 424/725; 514/44; 514/458; 514/465; 514/55; 514/725

(57) ABSTRACT

A pro-liposomal encapsulated product is made by the process comprising the steps of: (1) forming a first solution comprised of a biogenic active ingredient and a solvent; (2) contacting a lecithin and/or a phospholipid with the first solution to form a second solution; and (3) contacting the second solution with water to form a pro-liposomal encapsulated product. The pro-liposomes are used for the preparation of cosmetic or pharmaceutical preparations.

### FIELD OF THE INVENTION

The invention is in the field of cosmetics and relates to pro-liposomal encapsulated biogenic active ingredients, to a process for their preparation, and to the use of the pro-liposomes for the preparation of cosmetic or pharmaceutical preparations.

### PRIOR ART

The preparation of demanding cosmetic or pharmaceutical preparations, which are usually covered by the made-up word “cosmeceuticals”, often uses natural substances. These have, for example, antiinflammatory, anti-oxidative properties, are able to stimulate the growth of certain cells, to protect cells against damage by environmental influences or to block enzymes in a targeted manner. The use of such substances, such as, for example, nucleic acids or plant extracts, however, is often associated with formulation problems. This starts with the fact that there is no stable supply form which can be incorporated into an oil phase without problems, and extends to the lack of stability of the compositions, particularly under the influence of heat. It is likewise undesired that the active ingredients decompose, for example as a result of solar irradiation, thus losing, completely or partially, their advantageous properties.

The complex object of the present invention was accordingly to provide biogenic active ingredients in a novel supply form which can be readily formulated which permits the preparation of preparations with improved thermal and photostability. At the same time, the photostability of the substances should be improved.

### DESCRIPTION OF THE INVENTION

The present invention provides pro-liposomal encapsulated preparations obtainable by treating biogenic active ingredients in cosmetically acceptable solvents with lecithins and/or phospholipids. To clarify the term, it may be pointed out that the pro-liposomes do not contain water and only absorb water to form true liposomes when they are introduced into an aqueous environment. The pro-liposomal encapsulated biogenic active ingredients are therefore anhydrous by definition.

Surprisingly, it has been found that the biogenic active ingredients present in pro-liposomal form are easier to formulate than nonencapsulated products, and the particularly aqueous preparations prepared therewith are stable even at 40° C. over a long period without deposition of the substances occurring. In addition, improved photostability is observed.

Biogenic Active Ingredients

Biogenic active ingredients are to be understood as meaning, for example, tocopherol, tocopherol acetate, tocopherol palmitate, ascorbic acid, (deoxy)ribonucleic acid and fragmentation products thereof, chitosans, betaglucans, retinol, bisabolol, allantoin, phytantriol, panthenol, AHA acids, amino acids, ceramides, pseudoceramides, essential oils, plant extracts and vitamin complexes.

Lecithins and Phospholipids

The term “lecithins” is understood by the person skilled in the art as meaning those glycerophospholipids which form from fatty acids, glycerol, phosphoric acid and choline as a result of esterification. In the specialist world, lecithins are therefore also often referred to as phosphatidyl-cholines (PC) and conform to the general formula

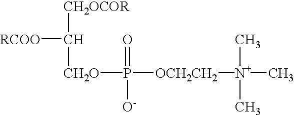

where R is typically a linear aliphatic hydrocarbon radical having 15 to 17 carbon atoms and up to 4 cis double bonds. Examples of natural lecithins suitable for the encapsulation which may be mentioned are the cephalines, which are also referred to phosphatidic acids and represent derivatives of 1,2-diacyl-sn-glycerol-3-phosphoric acids. By contrast, phospholipids are usually understood as meaning mono- and, preferably, diesters of phosphoric acid with glycerol (glycerol phosphates), which are generally assigned to the fats. In addition, sphingosines and sphingolipids are also suitable for the liposomal encapsulation. The use of lecithins and phospholipids for the preparation of liposomes is described, for example, by M. Schneider in Fat Sci. Technol. 94, 524 (1992) and U. Citernesi et al. in Cosm. Toil. 110, 57 (1995). In this connection, reference may also be made to the European patent specification EP 0525188 B1 (Takeda), from which liposomes are known whose shell membrane consists of nonionic surfactants and lecithins.

Process

The present invention further provides a process for the preparation of pro-liposomal encapsulated preparations in which biogenic active ingredients are treated in cosmetically acceptable solvents with lecithins and/or phospholipids. For this purpose, the biogenic active ingredients are usually initially introduced in a solvent and brought into contact with the lecithins or phospholipids at temperatures in the range from 30 to 70° C. The biogenic active ingredients and the lecithins and/or phospholipids can be used here in the weight ratio 1:20 to 5:1, preferably 1:2 to 4:1. Suitable solvents are preferably low alcohols having 1 to 4 carbon atoms, such as, for example, ethanol or polyols, which usually have 2 to 15 carbon atoms and at least two hydroxyl groups. The polyols can also contain further functional groups, in particular amino groups, or be modified with nitrogen. Typical examples are

glycerol;

alkylene glycols, such as, for example, ethylene glycol, diethylene glycol, propylene glycol, butylene glycol, hexylene glycol, and polyethylene glycols having an average molecular weight of from 100 to 1 000 daltons;

technical-grade oligoglycerol mixtures having a degree of self-condensation of from 1.5 to 10, such as, for example, technical-grade diglycerol mixtures having a diglycerol content of from 40 to 50% by weight;

methylol compounds, such as, in particular, trimethylolethane, trimethylolpropane, trimethylolbutane, pentaerythritol and dipentaerythritol;

lower alkyl glucosides, in particular those having 1 to 8 carbon atoms in the alkyl radical, such as, for example, methyl and butyl glucoside;

sugar alcohols having 5 to 12 carbon atoms, such as, for example, sorbitol or mannitol;

sugars having 5 to 12 carbon atoms, such as, for example, glucose or sucrose;

aminosugars, such as, for example, glucamine;

dialcoholamines, such as diethanolamine or 2-amino-1,3-propanediol.

In addition, it has proven advantageous to carry out the encapsulation in the presence of emulsifiers. Suitable for this purpose are, for example, nonionogenic surfactants from at least one of the following groups:

addition products of from 2 to 30 mol of ethylene oxide and/or 0 to 5 mol of propylene oxide to linear fatty alcohols having 8 to 22 carbon atoms, to fatty acids having 12 to 22 carbon atoms, to alkylphenols having 8 to 15 carbon atoms in the alkyl group, and alkylamines having 8 to 22 carbon atoms in the alkyl radical;

alkyl and/or alkenyl oligoglycosides having 8 to 22 carbon atoms in the alk(en)yl radical and the ethoxylated analogs thereof;

addition products of from 1 to 15 mol of ethylene oxide to castor oil and/or hydrogenated castor oil;

addition products of from 15 to 60 mol of ethylene oxide to castor oil and/or hydrogenated castor oil;

partial esters of glycerol and/or sorbitan with unsaturated, linear or saturated, branched fatty acids having 12 to 22 carbon atoms and/or hydroxy-carboxylic acids having 3 to 18 carbon atoms, and the adducts thereof with 1 to 30 mol of ethylene oxide;

partial esters of polyglycerol (average degree of self-condensation 2 to 8), polyethylene glycol (molecular weight 400 to 5 000), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and the adducts thereof with 1 to 30 mol of ethylene oxide;

mixed esters of pentaerythritol, fatty acids, citric acid and fatty alcohol as in German Patent 1165574 and/or mixed esters of fatty acids having 6 to 22 carbon atoms, methylglucose and polyols, preferably glycerol or polyglycerol,

mono-, di- and trialkyl phosphates, and mono-, di- and/or tri-PEG alkyl phosphates and salts thereof;

wool wax alcohols;

polysiloxane-polyalkyl-polyether copolymers and corresponding derivatives;

block copolymers, e.g. polyethylene glycol-30 dipolyhydroxystearates;

polymer emulsifiers, e.g. Pemulen grades (TR-1, TR-2) from Goodrich;

polyalkylene glycols, and

glycerol carbonate.

The addition products of ethylene oxide and/or of propylene oxide to fatty alcohols, fatty acids, alkylphenols or to castor oil are known, commercially available products. These are homolog mixtures whose average degree of alkoxylation corresponds to the ratio of the amounts of substance of ethylene oxide and/or propylene oxide and substrate with which the addition reaction is carried out. C_{12/18}-fatty acid mono- and diesters of addition products of ethylene oxide to glycerol are known from German Patent 2024051 as refatting agents for cosmetic preparations.

Alkyl and/or alkenyl oligoglycosides, their preparation and their use are known from the prior art. They are prepared, in particular, by reacting glucose or oligo-saccharides with primary alcohols having 8 to 18 carbon atoms. With regard to the glycoside radical, both monoglycosides, in which a cyclic sugar radical is glycosidically bonded to the fatty alcohol, and also oligomeric glycosides having a degree of oligomerization of up to, preferably, about 8, are suitable. The degree of oligomerization here is a statistical average value which is based on a homolog distribution customary for such technical-grade products.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid moglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride, and the technical-grade mixtures thereof which may also comprise small amounts of triglyceride as a minor product of the preparation process. Likewise suitable are addition products of 1 to 30 mol, preferably 5 to 10 mol, of ethylene oxide to said partial glycerides.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricin-oleate, sorbitan sesquiricinoleate, sorbitan diricin-oleate, sorbitan triricinoleate, sorbitan monohydroxy-stearate, sorbitan sesquihydroxystearate, sorbitan di-hydroxystearate, sorbitan trihydroxystearate, sorbitan monotartrate, sorbitan sesquitartrate, sorbitan di-tartrate, sorbitan tritartrate, sorbitan monocitrate, sorbitan sesquicitrate, sorbitan dicitrate, sorbitan tricitrate, sorbitan monomaleate, sorbitan sesqui-maleate, sorbitan dimaleate, sorbitan trimaleate, and technical-grade mixtures thereof. Likewise suitable are addition products of from 1 to 30 mol, preferably 5 to 10 mol, of ethylene oxide to said sorbitan esters.

Typical examples of suitable polyglycerol esters are polyglyceryl-2 dipolyhydroxystearate (Dehymuls® PGPH), polyglycerol-3 diisostearate (Lameform® TGI), poly-glyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (Polyglycerol Caprate T2010/90), polyglyceryl-3 cetyl ether (Chimexane® NL), polyglyceryl-3 distearate (Cremophor® GS 32) and polyglyceryl polyricinoleate (Admul® WOL 1403), poly-glyceryl dimerate isostearate, and mixtures thereof. Examples of further suitable polyol esters are the mono-, di- and triesters, optionally reacted with 1 to 30 mol of ethylene oxide, of trimethylolpropane or pentaerythritol with lauric acid, coconut fatty acid, tallow fatty acid, palmitic acid, stearic acid, oleic acid, behenic acid and the like.

Furthermore, zwitterionic surfactants can be used as emulsifiers. The term “zwitterionic surfactants” refers to those surface-active compounds which carry at least one quaternary ammonium group and at least one carboxylate and one sulfonate group in the molecule. Particularly suitable zwitterionic surfactants are the so-called betaines, such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethylhydroxyethylcarboxymethyl glycinate. Particular preference is given to the fatty acid amide derivative known under the CTFA name Cocamidopropyl Betaine. Likewise suitable emulsifiers are ampholytic surfactants. The term “ampholytic surfactants” means those surface-active compounds which, apart from a C_{8/18}-alkyl or -acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyl-iminodipropionic acids, N-hydroxyethyl-N-alkylamido-propylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacyl-aminoethylaminopropionate and C_{12/18}-acylsarcosine. Finally, cationic surfactants are also suitable emulsifiers, those of the ester quat type, preferably methyl-quaternized difatty acid triethanolamine ester salts, being particularly preferred. The amount of emulsifier can be 1 to 25% by weight, preferably 5 to 10% by weight, based on the biogenic active ingredients.

Industrial Applicability

The pro-liposomal encapsulated biogenic active ingredients according to the invention can be used for the preparation of cosmetic and/or pharmaceutical preparations, such as, for example, hair shampoos, hair lotions, foam baths, shower preparations, creams, gels, lotions, alcoholic and aqueous/alcoholic solutions, emulsions, wax/fatty compositions, stick preparations, powders or ointments in which they may be present in amounts of from 0.1 to 10% by weight, preferably 0.5 to 5% by weight and in particular 1 to 3% by weight, based on the final preparations.

These compositions can for their part also comprise, as further auxiliaries and additives, mild surfactants, oily substances, superfatting agents, pearlescent waxes, bodying agents, thickeners, polymers, silicone compounds, fats, waxes, stabilizers, deodorants, antiperspirants, antidandruff agents, film formers, swelling agents, UV light protection factors, antioxidants, preservatives, insect repellents, self-tanning agents, tyrosine inhibitors (depigmentation agents), solubilizers, perfume oils, dyes and the like. Nonencapsulated biogenic active ingredients and also emulsifiers and hydrotropic agents (lower alcohols and/or polyols), as already described in more detail above, may likewise be present.

Surfactants

Surface-active substances which may be present are anionic, nonionic, cationic and/or amphoteric or amphoteric surfactants, the content of which in the compositions is usually about 1 to 70% by weight, preferably 5 to 50% by weight and in particular 10 to 30% by weight. Typical examples of anionic surfactants are soaps, alkylbenzenesulfonates, alkanesulfonates, olefin sulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether)sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, protein fatty acid condensates (in particular wheat-based vegetable products) and alkyl (ether) phosphates. If the anionic surfactants contain polyglycolether chains, these may have a conventional homolog distribution, but preferably have a narrowed homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolysates (in particular vegetable products based on wheat), polyol fatty acid esters, sugar esters, sorbitan esters, polysorbates and amine oxides. If the nonionic surfactants contain polyglycol ether chains, these may have a conventional homolog distribution, but preferably have a narrowed homolog distribution. Typical examples of cationic surfactants are quaternary ammonium compounds, such as, for example, dimethyldistearylammonium chloride, and ester quats, in particular quaternized fatty acid trialkanolamine ester salts. Typical examples of amphoteric or zwitterionic surfactants are alkylbetains, alkylamidobetains, aminopropionates, aminoglycinates, imidazoliniumbetains and sulfobetains. Said surfactants are exclusively known compounds. With regard to structure and preparation of these substances, reference may be made to relevant review works, for example, J. Falbe (ed.), “Surfactants in Consumer Products”, Springer Verlag, Berlin, 1987, pp. 54-124 or J. Falbe (ed.), “Katalysatoren, Tenside und Mineralöladditive”, Thieme Verlag, Stuttgart, 1978, pp. 123-217. Typical examples of particularly suitable mild, i.e. particularly skin-compatible surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetains, amphoacetates and/or protein fatty acid condensates, the latter preferably based on wheat proteins.

Oily Substances

Suitable oily substances are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols and/or esters of branched C₆-C₁₃-carboxylic acids with linear or branched C₆-C₂₂-fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂-fatty acids with branched alcohols, in particular 2-ethylhexanol, esters of C₁₈-C₃₈-alkyl hydroxycarboxylic acids with linear or branched C₆-C₂₂-fatty alcohols (cf. DE 19756377 A1), in particular dioctyl malates, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, dicaprylyl carbonate (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or unsymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicon methicone types, inter alia) and/or aliphatic or naphthenic hydrocarbons, such as, for example, such as squalane, squalene or dialkylcyclohexanes.

Fats and Waxes

Typical examples of fats are glycerides, i.e. solid or liquid vegetable or animal products which consist essentially of mixed glycerol esters of higher fatty acids, suitable waxes are inter alia natural waxes, such as, for example, candelilla wax, carnauba wax, japan wax, esparto grass wax, cork wax, guaruma wax, rice germ oil wax, sugarcane wax, ouricury wax, montan wax, beeswax, shellac wax, spermaceti, lanolin (wool wax), uropygial grease, ceresin, ozokerite (earth wax), petrolatum, paraffin waxes, microcrystalline waxes; chemically modified waxes (hard waxes), such as, for example, montan ester waxes, sasol waxes, hydrogenated jojoba waxes, and synthetic waxes, such as, for example, polyalkylene waxes and polyethylene glycol waxes.

Pearlescent Waxes

Examples of suitable pearlescent waxes are: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have a total of at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Bodying Agents and Thickeners

Suitable bodying agents are primarily fatty alcohols or hydroxy fatty alcohols having 12 to 22, and preferably 16 to 18, carbon atoms, and also partial glycerides, fatty acids or hydroxy fatty acids. Preference is given to a combination of these substances with alkyl oligoglucosides and/or fatty acid N-methylglucamides of identical chain length and/or polyglycerol poly-12-hydroxystearates. Suitable thickeners are, for example, Aerosil grades (hydrophilic silicas), polysaccharides, in particular xanthan gum, guar guar, agar agar, alginates and Tyloses, carboxymethylcellulose and hydroxyethylcellulose, and also relatively high molecular weight polyethylene glycol mono- and diesters of fatty acids, polyacrylates (e.g. Carbopols® and Pemulen grades from Goodrich; Synthalens® from Sigma; Keltrol grades from Kelco; Sepigel grades from Seppic; Salcare grades from Allied Colloids), polyacrylamides, polymers, polyvinyl alcohol and polyvinylpyrrolidone, surfactants, such as, for example, ethoxylated fatty acid glycerides, esters of fatty acids with polyols such as, for example, pentaerythritol or trimethylolpropane, fatty alcohol ethoxylates having a narrowed homolog distribution or alkyl oligoglucosides, and electrolytes such as sodium chloride and ammonium chloride.

Superfatting Agents

Superfatting agents which can be used are substances such as, for example, lanolin and lecithin, and poly-ethoxylated or acylated lanolin and lecithin derivatives, polyol fatty acid esters, monoglycerides and fatty acid alkanolamides, the latter also serving as foam stabilizers.

Stabilizers

Stabilizers which can be used are metal salts of fatty acids, such as, for example, magnesium, aluminum and/or zinc stearate or ricinoleate.

Polymers

Suitable cationic polymers are, for example, cationic cellulose derivatives, such as, for example, a quaternized hydroxyethylcellulose obtainable under the name Polymer JR 400® from Amerchol, cationic starch, copolymers of diallylammonium salts and acrylamides, quaternized vinylpyrrolidone-vinylimidazole polymers, such as, for example, Luviquat® (BASF), condensation products of polyglycols and amines, quaternized collagen polypeptides, such as, for example, lauryldimonium hydroxypropyl hydrolyzed collagen (Lamequat® L/Grünau), quaternized wheat polypeptides, polyethyleneimine, cationic silicone polymers, such as, for example, amidomethicones, copolymers of adipic acid and dimethylaminohydroxypropyldiethylenetriamine (Cartaretins®/Sandoz), copolymers of acrylic acid with dimethyldiallylammonium chloride (Merquat® 550/ Chemviron), polyaminopolyamides, as described, for example, in FR 2252840 A, and crosslinked water-soluble polymers thereof, cationic chitin derivatives, such as, for example, quaternized chitosan, optionally in micro-crystalline dispersion, condensation products from dihaloalkyls, such as, for example, dibromobutane with bisdialkylamines, such as, for example, bis-dimethyl-amino-1,3-propane, cationic guar gum, such as, for example, Jaguar® CBS, Jaguar® C-17, Jaguar® C-16 from Celanese, quaternized ammonium salt polymers, such as, for example, Mirapol® A-15, Mirapol® AD-1, Mirapol AZ-1 from Miranol.

Suitable anionic, zwitterionic, amphoteric and nonionic polymers are, for example, vinyl acetate-crotonic acid copolymers, vinylpyrrolidone-vinyl acrylate copolymers, vinyl acetate-butyl maleate-isobornyl acrylate copolymers, methyl vinyl ether-maleic anhydride copolymers and esters thereof, uncrosslinked polyacrylic acids and polyacrylic acids crosslinked with polyols, acrylamido-propyltrimethylammonium chloride-acrylate copolymers, octylacrylamide-methyl methacrylate-tert-butylamino-ethyl methacrylate-2-hydroxypropyl methacrylate copolymers, polyvinylpyrrolidone, vinylpyrrolidone-vinyl acetate copolymers, vinylpyrrolidone-dimethylaminoethyl methacrylate-vinylcaprolactam terpolymers, and optionally derivatized cellulose ethers and silicones. Further suitable polymers and thickeners are listed in Cosm. Toil. 108, 95 (1993).

Silicone Compounds

Suitable silicone compounds are, for example, dimethyl-polysiloxanes, methylphenylpolysiloxanes, cyclic silicones, and amino-, fatty-acid-, alcohol-, polyether-, epoxy-, fluorine-, glycoside- and/or alkyl-modified silicone compounds, which can either be liquid or in resin form at room temperature. Also suitable are simethicones, which are mixtures of dimethicones having an average chain length of from 200 to 300 dimethyl-siloxane units and hydrogenated silicates. A detailed review of suitable volatile silicones can additionally be found in Todd et al., Cosm. Toil. 91, 27 (1976).

UV light Protection Filters and Antioxidants

UV light protection factors are, for example, to be understood as meaning organic substances (light protection filters) which are liquid or crystalline at room temperature and which are able to absorb ultraviolet rays and give off the absorbed energy again in the form of longer-wavelength radiation, e.g. heat. UVB filters can be oil-soluble or water-soluble. Examples of oil-soluble substances are:

3-benzylidenecamphor or 3-benzylidenenorcamphor and derivatives thereof, e.g. 3-(4-methylbenzylidene)-camphor, as described in EP 0693471 B1;

4-aminobenzoic acid derivatives, preferably 2-ethylhexyl 4-(dimethylamino)benzoate, 2-octyl 4-(dimethylamino)benzoate and amyl 4-(dimethylamino)-benzoate;

esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenyl-cinnamate (octocrylene);

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methyl-benzophenone, 2,2′-dihydroxy-4-methoxybenzophenone;

esters of benzalmalonic acid, preferably di-2-ethyl-hexyl 4-methoxybenzmalonate;

triazine derivatives, such as, for example, 2,4,6-trianilino(p-carbo-2′-ethyl-1′-hexyloxy)-1,3,5-triazine and octyltriazone, as described in EP 0818450 A1 or dioctylbutamidotriazone (Uvasorb® HEB);

propane-1,3-diones, such as, for example, 1-(4-tert-butylphenyl)-3-(4′-methoxyphenyl)propane-1,3-dione;

ketotricyclo(5.2.1.0)decane derivatives, as described in EP 0694521 B1.

Suitable water-soluble substances are:

2-phenylbenzimidazole-5-sulfonic acid and the alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and its salts;

sulfonic acid derivatives of 3-benzylidenecamphor, such as, for example, 4-(2-oxo-3-bornylidenemethyl)-benzenesulfonic acid and 2-methyl-5-(2-oxo-3-bornyl-idene)sulfonic acid and salts thereof.

Suitable typical UV-A filters are, in particular, derivatives of benzoylmethane, such as, for example, 1-(4′-tert-butylphenyl)-3-(4′-methoxyphenyl)propane-1,3-dione, 4-tert-butyl-4′-methoxydibenzoylmethane (Parsol 1789), 1-phenyl-3-(4′-isopropylphenyl)propane-1,3-dione, and enamine compounds, as described in DE 19712033 A1 (BASF). The UV-A and UV-B filters can of course also be used in mixtures. Particularly favorable compositions consist of the derivatives of benzoyl-methane, e.g. 4-tert-butyl-4′-methoxydibenzoylmethane (Parsol® 1789) and 2-ethylhexyl 2-cyano-3,3-phenyl-cinnamate (octocrylene) in combination with esters of cinnimic acid, preferably 2-ethylhexyl 4-methoxy-cinnamate and/or propyl 4-methoxycinnamate and/or isoamyl 4-methoxycinnamate. Advantageously, such combinations are combined with water-soluble filters such as, for example, 2-phenylbenzimidazole-5-sulfonic acid and their alkali metal, alkaline earth metal, ammonium, alkylammonium, alkanolammonium and glucammonium salts.

As well as said soluble substances, insoluble light protection pigments, namely finely dispersed metal oxides or salts, are also suitable for this purpose. Examples of suitable metal oxides are, in particular, zinc oxide and titanium oxide and also oxides of iron, zirconium, silicon, manganese, aluminum and cerium, and mixtures thereof. Salts which may be used are silicates (talc), barium sulfate or zinc stearate. The oxides and salts are used in the form of the pigments for skincare and skin-protective emulsions and decorative cosmetics. The particles here should have an average diameter of less than 100 nm, preferably between 5 and 50 nm and in particular between 15 and 30 nm. They can have a spherical shape, but it is also possible to use particles which have an ellipsoidal shape or a shape deviating in some other way from the spherical form. The pigments can also be surface-treated, i.e. hydrophilicized or hydrophobicized. Typical examples are coated titanium dioxides, such as, for example, titanium dioxide T 805 (Degussa) or Eusolex® T2000 (Merck). Suitable hydrophobic coating agents are here primarily silicones and, specifically in this case, trialkoxyoctylsilanes or simethicones. In sunscreens, preference is given to using so-called micro- or nanopigments. Preference is given to using micronized zinc oxide. Further suitable UV light protection filters are given in the review by P. Finkel in SÖFW-Journal 122, 543 (1996) and Parf. Kosm. 3, 11 (1999).

As well as the two abovementioned groups of primary light protection substances, it is also possible to use secondary light protection agents of the antioxidant type; these interrupt the photochemical reaction chain which is triggered when UV radiation penetrates the skin. Typical examples thereof are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chloro-genic acid and derivatives thereof, lipoic acid and derivatives thereof (e.g. dihydrolipoic acid), auro-thioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of gum benzoin, rutic acid and derivatives thereof, α-glycosylrutin, ferulic acid, furfurylidene-glucitol, carnosine, butylhydroxytoluene, butylhydroxy-anisole, nordihydroguaiacic acid, nordihydroguaiaretic acid, trihydroxybutyrophenone, uric acid and derivatives thereof, mannose and derivatives thereof, super-oxide dismutase, zinc and derivatives thereof (e.g. ZnO, ZnSO₄) selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention. Preference is given here primarily to mixtures of carotinoids and hydroxycarboxylic acids.

Deodorants and Antimicrobial Agents

Cosmetic deodorants counteract, mask or remove body odors. Body odors arise as a result of the effect of skin bacteria on apocrine perspiration, with the formation of degradation products which have an unpleasant odor. Accordingly, deodorants comprise active ingredients which act as antimicrobial agents, enzyme inhibitors, odor absorbers or odor masking agents. Suitable antimicrobial agents are, in principle, all substances effective against gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, oil of cloves, menthol, mint oil, farnesol, phenoxy-ethanol, glycerol monocaprate, glycerol monocaprylate, glycerol monolaurate (GML), diglycerol monocaprate (DMC), salicylic acid N-alkylamides, such as, for example, n-octylsalicylamide or n-decylsalicylamide.

Suitable enzyme inhibitors are, for example, esterase inhibitors. These are preferably trialkyl citrates, such as trimethyl citrate, tripropyl citrate, triiso-propyl citrate, tributyl citrate and, in particular, triethyl citrate (Hydagen® CAT). The substances inhibit enzyme activity, thereby reducing the formation of odor. Other substances which are suitable esterase inhibitors are sterol sulfates or phosphates, such as, for example, lanosterol, cholesterol, campesterol, stigmasterol and sitosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

Suitable odor absorbers are substances which are able to absorb and largely retain odor-forming compounds. They lower the partial pressure of the individual components, thus also reducing their rate of diffusion. It is important that in this process perfumes must remain unimpaired. Odor absorbers are not effective against bacteria. They comprise, for example, as main constituent, a complex zinc salt of ricinoleic acid or specific, largely odor-neutral fragrances which are known to the person skilled in the art as “fixatives”, such as, for example, extracts of labdanum or styrax or certain abietic acid derivatives. The odor masking agents are fragrances or perfume oils, which, in addition to their function as odor masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethol, citronellol, eugenol, isoeugenol, geraniol, linalool, phenylethyl alcohol and terpineol, and the hydrocarbons include mainly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinnamon leaf oil, linden flower oil, juniper berry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzyl-acetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Antiperspirants reduce the formation of perspiration by influencing the activity of the eccrine sweat glands, thus counteracting underarm wetness and body odor. Aqueous or anhydrous formulations of antiperspirants typically comprise the following ingredients:

astringent active ingredients,

oil components,

nonionic emulsifiers,

coemulsifiers,

bodying agents,

auxiliaries, such as, for example, thickeners or complexing agents and/or

nonaqueous solvents, such as, for example, ethanol, propylene glycol and/or glycerol.

Suitable astringent antiperspirant active ingredients are primarily salts of aluminum, zirconium or of zinc. Such suitable antihydrotic active ingredients are, for example, aluminum chloride, aluminum chlorohydrate, aluminum dichlorohydrate, aluminum sesquichlorohydrate and complex compounds thereof, e.g. with 1,2-propylene glycol, aluminum hydroxyallantoinate, aluminum chloride tartrate, aluminum zirconium trichlorohydrate, aluminum zirconium tetrachlorohydrate, aluminum zirconium penta-chlorohydrate and complex compounds thereof, e.g. with amino acids, such as glycine. In addition, customary oil-soluble and water-soluble auxiliaries may be present in antiperspirants in relatively small amounts. Such oil-soluble auxiliaries may, for example, be:

anti-inflammatory, skin-protective or perfumed essential oils,

synthetic skin-protective active ingredients and/or

oil-soluble perfume oils.

Customary water-soluble additives are, for example, preservatives, water-soluble fragrances, pH regulators, e.g. buffer mixtures, water-soluble thickeners, e.g. water-soluble natural or synthetic polymers, such as, for example, xanthan gum, hydroxyethylcellulose, polyvinylpyrrolidone or high molecular weight polyethylene oxides.

Film Formers

Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid and salts thereof, and similar compounds.

Antidandruff Active Ingredients

Suitable antidandruff active ingredients are pirocton olamin (1-hydroxy-4-methyl-6-(2,4,4-trimethylpentyl)-2-(1H)-pyridinone monoethanolamine salt), Baypival® (climbazole), Ketoconazole®, (4-acetyl-1-{-4-[2-(2,4-dichlorophenyl) r-2-(1H-imidazol-1-ylmethyl)-1,3-dioxylan-c-4-ylmethoxyphenyl}piperazine, ketocona-zole, elubiol, selenium disulfide, sulfur colloidal, sulfur polyethylene glycol sorbitan monooleate, sulfur ricinole polyethoxylate, sulfur tar distillates, salicyclic acid (or in combination with hexachloro-phene), undecylenic acid monoethanolamide sulfosuccinate Na salt, Lamepon® UD (protein undecylenic acid condensate), zinc pyrithione, aluminum pyrithione and magnesium pyrithione/dipyrithione magnesium sulfate.

Swelling Agents

The swelling agents for aqueous phases may be montmorillonites, clay mineral substances, Pemulen, and alkyl-modified Carbopol grades (Goodrich). Other suitable polymers and swelling agents are given in the review by R. Lochhead in Cosm. Toil. 108, 95 (1993).

Insect Repellents

Suitable insect repellents are N,N-diethyl-m-toluamide, 1,2-pentanediol or ethyl butylacetylaminopropionate.

Self-tanning Agents and Depigmentation Agents

A suitable self-tanning agent is dihydroxyacetone. Suitable tyrosine inhibitors, which prevent the formation of melanin and are used in depigmentation agents, are, for example, arbutin, ferulic acid, kojic acid, coumaric acid and ascorbic acid (vitamin C).

Preservatives

Suitable preservatives are, for example, phenoxy ethanol, formaldehyde solution, parabenes, pentanediol or sorbic acid, and the other classes of substance listed in Annex 6, Part A and B of the Cosmetics Directive.

Perfume Oils

Perfume oils which may be mentioned are mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamon, costus, iris, calmus), woods (pine wood, sandalwood, guaiac wood, cedarwood, rosewood), herbs and grasses (tarragon, lemon grass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl iso-butyrate, p-tert-butylcyclohexyl acetate, linalyl acetate, dimethylbenzylcarbinyl acetate, phenylethyl acetate, linalyl benzoate, benzyl formate, ethylmethyl-phenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include predominantly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzyl-acetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Dyes

Dyes which can be used are the substances which are approved and suitable for cosmetic purposes, as are summarized, for example, in the publication “Kosmetische Färbemittel” [Cosmetic Colorants] from the Farbstoffkommission der Deutschen Forschungsgemeinschaft [Dyes Commission of the German Research Council], Verlag Chemie, Weinheim, 1984, pp. 81-106. These dyes are normally used in concentrations of from 0.001 to 0.1% by weight, based on the total mixture.

The total amount of auxiliaries and additives can be 1 to 50% by weight, preferably 5 to 40% by weight, based on the compositions. The compositions can be prepared by customary cold or hot processes; preference is given to using the phase-inversion temperature method.

### EXAMPLES

### Example P1

10 g of deoxyribonucleic acid, 21.5 g of propylene glycol, 3 g of sorbitan monostearate+20EO (Eumulgin® SMS 20, Cognis Deutschland GmbH) and 0.5 g of preservative (Phenonip) were initially introduced into a stirred apparatus, and 65 g of pulverulent soya lecithin (weight ratio of oily bodies: lecithin=1:6.5) were added. The mixture was heated to 65° C. until a homogeneous, clear solution resulted and was then filtered in order to separate off undissolved lecithin. Introducing the mixture into water gave liposomes with an average diameter of 150 nm.

### Example P2

A mixture of 22 g of chitosan (Hydagen® CMF, Cognis Deutschland GmbH) and 5 g of cocoglucosides (Plantcare® APG 1200, Cognis Deutschland GmbH) was initially introduced into a stirred apparatus, and 44 g of pulverulent soya lecithin (weight ratio of oily bodies:lecithin=1:2) were added. The mixture was diluted by adding 33 g of propylene glycol, heated to 65° C. and stirred until a homogeneous, clear solution resulted and was then filtered in order to separate off undissolved lecithin. Introducing the mixture into water gave liposomes with an average diameter of 150 nm.

### Example P3

A mixture of 20 g of bisabolol and 5 g of cocoglucosides (Plantacare® APG 1200, Cognis Deutschland GmbH) was initially introduced into a stirred apparatus, and 60 g of pulverulent soya lecithin (weight ratio of oily bodies:lecithin=1:3) were added. The mixture was diluted by adding 33 g of dipropylene glycol, heated to 65° C. and stirred until a homogeneous, clear solution resulted and was then filtered in order to separate off undissolved lecithin. Introducing the mixture into water gave liposomes with an average diameter of 150 nm.

### Example P4

A mixture of 50 g of retinol and 5 g of sorbitan sesquioleate+20EO (Eumulgin® SSO 20, Cognis Deutschland GmbH) were initially introduced into a stirred apparatus, and 50 g of pulverulent soya lecithin (weight ratio of oily bodies:lecithin=1:1) were added. The mixture was diluted by adding 35 g of glycerol, heated to 65° C. and stirred until a homogeneous, clear solution resulted and was then filtered in order to separate off undissolved lecithin. Introducing the mixture into water gave liposomes with an average diameter of 150 nm.

Performance investigations.

Aqueous preparations with different oily bodies and pro-liposomal or non-encapsulated biogenic active ingredients were prepared and their stability upon storage and under the influence of temperature was investigated. The results are summarized in Table 1. (+) means stable, (−) unstable, i.e. deposition of the filter. Examples 1 to 6 are in accordance with the invention, and Examples C1 to C6 are used as a comparison.

TABLE 1
Stability of skincare compositions
(quantitative data as % by weight)

| Composition | 1 | 2 | 3 | 4 | 5 | 6 | C1 | C2 | C3 | C4 | C5 | C6 |
|---|---|---|---|---|---|---|---|---|---|---|---|---|
| Squalane | 15 | 15 | 15 | 15 | − | − | 15 | 15 | 15 | 15 | − | − |
| Dicaprylyl | − | − | − | − | 15 | 15 | − | − | − | − | 15 | 15 |
| carbonate | | | | | | | | | | | | |
| Polyglycerol - | 5 | 5 | 5 | 5 | 5 | 5 | 5 | 5 | 5 | 5 | 5 | 5 |
| 2 | | | | | | | | | | | | |
| dipoly- | | | | | | | | | | | | |
| hydroxy - | | | | | | | | | | | | |
| stearate | | | | | | | | | | | | |
| Deoxyribo- | 3 | − | − | − | 3 | − | − | − | − | − | − | − |
| nucleic acid | | | | | | | | | | | | |
| (P1) | | | | | | | | | | | | |
| Chitosan (P2) | − | 3 | − | − | − | 3 | − | − | − | − | − | − |
| Bisabolol (P3) | − | − | 3 | − | − | − | − | − | − | − | − | − |
| Retinol (P4) | − | − | − | 3 | − | − | − | − | − | − | − | − |
| Deoxyribo- | − | − | − | − | − | − | 3 | − | − | − | 3 | − |
| nucleic acid | | | | | | | | | | | | |
| Chitosan | − | − | − | − | − | − | − | 3 | − | − | − | 3 |
| Bisabolol | − | − | − | − | − | − | − | − | 3 | − | − | − |
| Retinol | − | − | − | − | − | − | − | − | − | 3 | − | − |
| Water | ad 100 | | | | | | | | | | | |
| Stability | | | | | | | | | | | | |
| Immediately | + | + | + | + | + | + | + | + | + | + | + | + |
| After 1 week, | + | + | + | + | + | + | + | − | + | + | + | + |
| (20° C.) | | | | | | | | | | | | |
| After 4 weeks, | + | + | + | + | + | − | − | − | − | − | − | − |
| (40° C.) | | | | | | | | | | | | |

Table 2 gives a number of formulation examples for skincare compositions using the pro-liposomal encapsulated biogenic active ingredients.

TABLE 2
Cosmetic preparations (water,
preservatives ad 100% by weight)

| Composition | | | | | | | | | | |
|---|---|---|---|---|---|---|---|---|---|---|
| (INCI) | 1 | 2 | 3 | 4 | 5 | 6 | 7 | 8 | 9 | 10 |
| Emulgade ® SE | 5.0 | 5.0 | 5.0 | 4.0 | 5.0 | 4.0 | — | — | — | — |
| Glyceryl stearate | | | | | | | | | | |
| (and) ceteareth | | | | | | | | | | |
| 12/20 (and) cetyl | | | | | | | | | | |
| palmitate | | | | | | | | | | |
| Eumulgin ® B1 | — | — | — | 1.0 | — | 1.0 | — | — | — | — |
| Ceteareth - 12 | | | | | | | | | | |
| Lameform ® TGI | — | — | — | — | — | — | 4.0 | — | 4.0 | — |
| Polyglyceryl - 3 | | | | | | | | | | |
| isostearate | | | | | | | | | | |
| Dehymuls ® PGPH | — | — | — | — | — | — | — | 4.0 | — | 4.0 |
| Polyglyceryl - 2 | | | | | | | | | | |
| dipolyhydroxy - | | | | | | | | | | |
| stearate | | | | | | | | | | |
| Monomulus ® | — | — | — | — | — | — | 2.0 | — | 2.0 | — |
| 90 - O 18 | | | | | | | | | | |
| Glyceryl oleate | | | | | | | | | | |
| Cetiol ® HE | — | — | — | — | — | — | — | 2.0 | — | 2.0 |
| PEG - 7 glyceryl | | | | | | | | | | |
| cocoate | | | | | | | | | | |
| Cetiol ® SQ | 3.0 | — | 3.0 | 3.0 | — | 4.0 | 5.0 | 5.0 | 5.0 | 5.0 |
| Squalane | | | | | | | | | | |
| Cetiol ® OE | 3.0 | 1.0 | — | — | — | 1.0 | 5.0 | 6.0 | — | — |
| Dicaprylyl ether | | | | | | | | | | |
| Cetiol ® CC | — | 3.0 | 4.0 | — | 4.0 | 2.0 | — | — | 5.0 | 6.0 |
| Dicaprylyl | | | | | | | | | | |
| carbonate | | | | | | | | | | |
| Cetiol ® PGL | — | — | — | 3.0 | — | — | 10.0 | 9.0 | 10.0 | 9.0 |
| Hexyldecanol | | | | | | | | | | |
| (and) hexyldecyl | | | | | | | | | | |
| laurate | | | | | | | | | | |
| Cetiol ® V | 3.0 | 3.0 | 3.0 | — | 4.0 | — | — | — | — | — |
| Decyl oleate | | | | | | | | | | |
| Beeswax | — | — | — | — | — | — | 7.0 | 5.0 | 7.0 | 5.0 |
| Nutrilan ® Elastin | 2.0 | 2.0 | — | — | — | — | — | — | — | — |
| E20 | | | | | | | | | | |
| Hydrolyzed | | | | | | | | | | |
| elastin | | | | | | | | | | |
| Nutrilan ® I-50 | — | — | 2.0 | — | 2.0 | — | — | — | — | — |
| Hydrolyzed | | | | | | | | | | |
| collagen | | | | | | | | | | |
| Gluadin ® AGP | — | — | — | 0.5 | — | 0.5 | — | — | — | — |
| Hydrolyzed wheat | | | | | | | | | | |
| gluten | | | | | | | | | | |
| Gluadin ® WK | — | — | — | — | — | — | 0.5 | 0.5 | 0.5 | 0.5 |
| Sodium cocoyl | | | | | | | | | | |
| hydrolyzed wheat | | | | | | | | | | |
| protein | | | | | | | | | | |
| Arlyon ® F | — | 2.0 | — | — | 2.0 | 2.0 | — | — | 2.0 | 2.0 |
| Laureth - 2 | | | | | | | | | | |
| Highcareen ® GS | 1.0 | 1.0 | 1.0 | 1.0 | 1.0 | 1.0 | 1.0 | 1.0 | 1.0 | 1.0 |
| pro - liposomes | | | | | | | | | | |
| Betaglucan | | | | | | | | | | |
| Hydagen ® CMF | 1.0 | 1.0 | 1.0 | 1.0 | 1.0 | 1.0 | 1.0 | 1.0 | 1.0 | 1.0 |
| pro - liposomes | | | | | | | | | | |
| Chitosan | | | | | | | | | | |
| Magnesium sulfate | — | — | — | — | — | — | 1.0 | 1.0 | 1.0 | 1.0 |
| hepta hydrate | | | | | | | | | | |
| Glycerol (86% | 3.0 | 3.0 | 3.0 | 5.0 | 3.0 | 5.0 | 5.0 | 3.0 | 5.0 | 3.0 |
| strength by | | | | | | | | | | |
| weight) | | | | | | | | | | |

(1,2) soft cream, (3-6) moisturizing emulsion, (7-10) night cream

1. A pro-liposomal encapsulated preparation obtainable by treating biogenic active ingredients in cosmetically acceptable solvents with lecithins and/or phospholipids.

2. A process for the preparation of pro-liposomal encapsulated preparations in which biogenic active ingredients are treated in cosmetically acceptable solvents with lecithins and/or phospholipids.

3. The process as claimed in claim 2, characterized in that biogenic active ingredients are used which are chosen from the group formed by tocopherol, tocopherol acetate, tocopherol palmitate, ascorbic acid, (deoxy)ribonucleic acid and fragmentation products thereof, chitosans, betaglucans, retinol, bisabolol, allantoin, phytantriol, panthenol, AHA acids, amino acids, ceramides, pseudoceramides, essential oils, plant extracts and vitamin complexes.

4. The process as claimed in claims 2 and/or 3, characterized in that the biogenic active ingredients and the lecithins and/or phospholipids are used in the weight ratio 1:20 to 5:1.

5. The process as claimed in at least one of claims 2 to 4, characterized in that the solvents used are lower alcohols having 1 to 4 carbon atoms and/or polyols.

6. The process as claimed in at least one of claims 2 to 5, characterized in that solvents are used which are chosen from the group formed by ethanol, ethylene glycol, propylene glycol, butylene glycol, polyethylene glycol with molar weights in the range from 100 to 1 000 and glycerol.

7. The process as claimed in at least one of claims 2 to 6, characterized in that the encapsulation is carried out in the presence of emulsifiers.

8. The use of pro-liposomal encapsulated preparations as claimed in claim 1 of the preparation of cosmetic and/or pharmaceutical preparations.
